(19) United States
(12) Patent Application Publication
(10) Pub. No.: US 2006/0253302 A1
(43) Pub. Date: Nov. 9, 2006

(54) SYSTEM FOR FACILITATING HEALTHCARE SERVICES FOR CASH PAYING PATIENTS

(75) Inventor: Jeffrey Arthur Oster, Granville, OH (US)

Correspondence Address: WOOD, HERRON & EVANS, LLP, 2700 CAREW TOWER, 441 VINE STREET, CINCINNATI, OH, 45202, US

(73) Assignee: Jeffrey Arthur Oster, Granville, OH (US)

(21) Appl. No.: 11/381,423

(22) Filed: May 3, 2006

Related U.S. Application Data
(60) Provisional application No. 60/677,221, filed on May 3, 2005.

(51) Int. Cl. G06Q 10/00; A61B 5/00

(52) U.S. Cl. 705/2; 600/300

(57) ABSTRACT

An online system and process include an Internet search engine and a database used to arrange services between healthcare providers and uninsured or other cash paying patients. The database may include healthcare information pertaining to doctors and dentists who will accept patients having no health insurance and/or paying with cash. Patient searches are typically performed by zip code and provide a doctor profile that describes the doctor, their office policies and fees for common office services.

### CROSS REFERENCE TO RELATED APPLICATIONS

This application claims benefit of priority to Provisional Patent Application No. 60/677,221, entitled “SYSTEM FOR COORDINATING HEALTHCARE FOR UNINSURED PATIENTS”, filed May 3, 2005 by Jeffrey Arthur Oster, which application is incorporated herein by reference in its entirety.

### FIELD OF THE INVENTION

The present invention generally relates to health care services, and more particularly, to facilitating patient and doctor health service transactions.

### BACKGROUND OF THE INVENTION

In response to the growing number of uninsured patients, physicians are becoming aware of a new cash paying market. Payment by check, credit card, currency, debit card, coin, note and other cash tender is increasing as societal changes place more people in a position where they can no longer carry healthcare benefits. Government and industry trends, among other factors, contribute to a growing number of uninsured individuals and families for whom traditional healthcare coverage is out of reach. For instance, independent contractors, laid-off workers, divorced single mothers, college students, small business owners, the working poor and their children must routinely forego health insurance to meet basic needs.

In addition to uninsured patients, some healthcare providers now encourage cash payment for services using Health Savings Accounts (HSA's). A HSA is a tax advantaged savings plan that allows insurers to deposit money to pay for current and future medical expenses. Money can be deposited to a HSA before tax is paid on it. The money can be used tax-free for medical expenses, chiefly one's medical insurance deductible and co-insurance. The account is otherwise similar to a traditional bank account in that checks can be written against the account. By including elements of a conventional cash transactions, HSA's encourage participants to spend their allocated funds conscientiously.

Despite inherent advantages to both patients and doctors in conducting cash transactions, there is no structured, effective and/or large scale mechanism for efficiently bringing together healthcare service providers and cash paying patients. There consequently exists a need for an improved manner of facilitating patient and doctor healthcare service transactions.

### SUMMARY OF THE INVENTION

The present invention provides a computer implemented method and apparatus for facilitating healthcare services between healthcare providers and cash paying patients. To this end, embodiments of the invention receive from a cash paying patient a user preference relating to a desired healthcare service. A database is accessed to correlate the user preference to healthcare provider information. One or more healthcare providers associated with matched healthcare provider information may be identified and displayed to the patient.

The user preference typically includes at least one of a geographic, price, biographic or service-related preference. As such, the user preference will generally correspond to the type of data comprising the healthcare provider information, as input and routinely updated by the respective healthcare providers. Typical cash transactions facilitated by such features include check, coin, note, credit card, debit card and HSA payments.

In this manner, aspects of the invention enable healthcare providers to capitalize on the emerging, cash-paying market. In addition to expanding the pool of potential patients, cash transactions may actually cost less in time and money than providing care to patients paying with insurance.

Other benefits to doctors may include no waiting for payments, no prior authorizations, no determination of co-payments and no retroactive payment denials. Still other advantages include less time spent in correspondence with insurers, fewer accounts to send to collections and access to a new and growing patient population.

Free from the conventional constraints of insurance contracts, doctors may be able to set a more flexible and mutually beneficial fee for services. Knowing that it can cost less to provide care in a cash for services rendered practice, doctors may be able to pass on substantial savings to their cash paying patients.

Other benefits include prompt access to care that results in early intervention of illness, thereby reducing the severity of an illness. The severity of an illness is often directly proportional to the cost of care. Early intervention often lowers the cost of providing healthcare. Prompt access to care also decreases costly crisis care in the emergency room.

These and other advantages and features that characterize the invention are set forth in the claims annexed hereto and forming a further part hereof. However, for a better understanding of the invention, and of the advantages and objectives attained through its use, reference should be made to the Drawings, and to the accompanying descriptive matter, in which there is described exemplary embodiments of the invention.

### BRIEF DESCRIPTION OF THE DRAWINGS

FIG. 1 is a block diagram of a networked computer system configured to facilitate healthcare services between providers and cash paying patients.

FIG. 2 is flowchart having steps suitable for execution by the computer system of FIG. 1 to determine and display healthcare providers appropriate for a cash paying user.

FIG. 3 is flowchart having steps suitable for execution by the computer system of FIG. 1 for receiving and posting healthcare provider information in a searchable format to facilitate provider determination.

### DETAILED DESCRIPTION

An embodiment consistent with the invention provides an online system and process that includes both an Internet search engine and a database used to arrange services between healthcare providers and cash paying patients. The database may include healthcare information pertaining to healthcare providers who will accept patients having no health insurance and/or paying with cash. Online patient searches are typically performed using keywords, e.g., a zip code, and provide a doctor profile including a description of the doctor, their office policies and fees for common office services.

To this end, the system typically includes an Internet-based search engine that enables cash paying patients to access doctors who are interested in seeing them in their offices for primary care services. Such cash transactional users may include patients paying for services using cash, check, debit card, credit card, or a HSA, among others. One group that particularly relies upon such cash transactions includes uninsured patients.

The system may provide a profile on a web page for healthcare providers that includes their practice, the doctor's training, and itemizes a number of the most common services provided by that healthcare provider. The services are typically a small sample of the many different services provided by that healthcare provider. Exemplary healthcare providers may include M.D.'s, D.O.'s, D.P.M.'s, D.C.'s and D.D.S.'s.

In addition to providing users with the most appropriate demographic, service and geographic healthcare providers, users typically may view an amount posted by healthcare provider that will be charged to a patient who is uninsured or is otherwise desiring a cash transaction. This charged amount will typically be less than an amount charged to an insured user because of the overhead associated with the administration of a claim, including billing, collection, coverage verification, etc.

When patients view the website, they may initially be prompted to enter their zip code. The system 10 may search its provider database 37 and provide search results ranked by zip code (nearest zip code first). More in-depth searches may include the specialty of a provider, among other user preferences.

Users may print the page of the doctor that they wish to see and may call for an appointment. Patients print the page so that they may sign a disclaimer to be kept at the doctor's office. The disclaimer typically states that the patient is currently uninsured. This disclaimer may protect the doctor from patients who are actually insured, but who choose to use the system's web services. This disclaimer may further protect the doctor from any breach of contract that he or she may have in existence with an insurance company. Another reason to print the doctor profile page is to have a record of the fees that the uninsured patient will be charged by the doctor. Doctors may have constant access to their web page and may update information or change fees as needed.

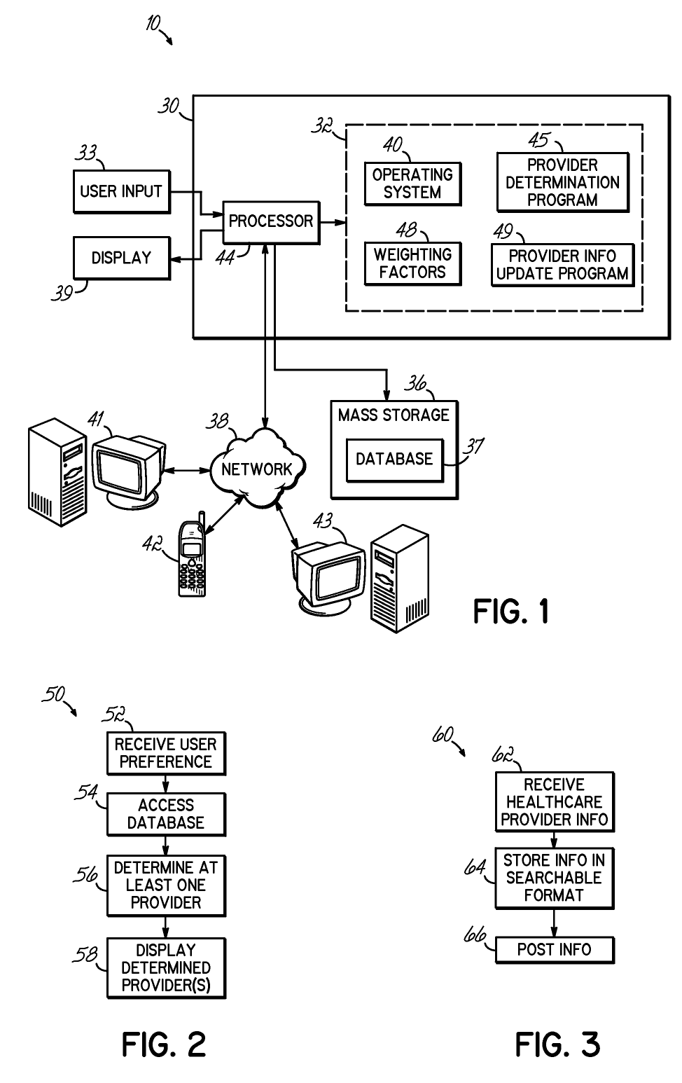

While the principles of this invention do not limit its forum or application, one desirable embodiment capitalizes on the structure available through the computer networked systems exemplified in FIG. 1. FIG. 1 generally shows a block diagram of a networked computer system 10 configured to facilitate healthcare services between providers and cash paying patients. The system 10 more particularly comprises one or more client computer(s) 30 coupled to a network 38. Network 38 represents a networked interconnection, including, but not limited to local-area, wide-area, wireless, and public networks (e.g., the Internet). Moreover, any number of computers and other devices may be networked through network 38, e.g., multiple servers. For instance, network 38 communicates with patient and provider electronic communication devices 41, 42 43.

Computer system 10 will hereinafter also be referred to as a “apparatus”, “computer”, or “healthcare coordination system”, although it should be appreciated that the terms may respectively include many other controller configurations. Moreover, while only a few network interface devices 30, 41, 42, 43 are shown in FIG. 1, any number of computers and other devices may be networked through network 38. In still another embodiment, the system 10 may be implemented in a stand-alone configuration, i.e., disconnected from another computer or computer network.

Computer 30 typically includes at least one processor 44 coupled to a memory 32. Processor 44 may represent one or more processors (e.g., microprocessors), and memory 32 may represent the random access memory (RAM) devices comprising the main storage of computer 30, as well as any supplemental levels of memory, e.g., cache memories, non-volatile or backup memories (e.g., programmable or flash memories), read-only memories, etc. In addition, memory 32 may be considered to include memory storage physically located elsewhere in computer 30, e.g., any cache memory present in processor 44, as well as any storage capacity used as a virtual memory, e.g., as stored within a database 37, or on another computer coupled to computer 30 via network 38.

Computer 30 also may receive a number of inputs and outputs for communicating information externally. For interface with a user, computer 30 typically includes one or more input devices 33 (e.g., a keyboard, a mouse, a trackball, a joystick, a touch pad, iris/fingerprint scanner, and/or a microphone, among others). The computer 30 additionally includes a display 39 (e.g., a CRT monitor, an LCD display panel, and/or a speaker, among others). It should be appreciated, however, that with some implementations of the computer 30, direct user input and output may be unsupported by the computer, and interface with the server computer 30 may be implemented through a computer or workstation networked with the computer 30.

For additional storage, computer 30 may also include one or more mass storage devices 36 configured to store, for instance, the database 37. Exemplary devices 36 can include: a floppy or other removable disk drive, a flash drive, a hard disk drive, a direct access storage device (DASD), an optical drive (e.g., a CD drive, a DVD drive, etc.), and/or a tape drive, among others. Furthermore, computer 30 may include an interface with one or more networks (e.g., a LAN, a WAN, a wireless network, and/or the Internet, among others) to permit the communication of information with other computers coupled to the network 38. It should be appreciated that computer 30 typically includes suitable analog and/or digital interfaces between processor 44 and each of components 32, 33, 36, 38 and 39.

Computer 30 operates under the control of an operating system 40, and executes various computer software applications, components, programs, modules, e.g., a provider determination program 45, search engine weighting factors 48 and a provider information update program 44, among others. Various applications, components, programs, markers, modules, etc. may also execute on one or more processors in another computer coupled to computer 30 via a network 38, e.g., in a distributed or client-server computing environment, whereby the processing required to implement the functions of a computer program may be allocated to multiple computers over a network.

In general, the routines executed to implement the embodiments of the invention, whether implemented as part of an operating system or a specific application, component, program, engine, process, programmatic tool, object, module or sequence of instructions, or even a subset thereof, may be referred to herein as “computer program code”, or simply “program code”. Program code typically comprises one or more instructions that are resident at various times in various memory and storage devices in a computer, and that, when read and executed by one or more processors in a computer, cause that computer to perform the steps necessary to execute steps or elements embodying the various aspects of the invention. One of skill in the art should appreciate that embodiments consistent with the principles of the present invention may nonetheless use program code resident at only one, or any number of locations.

Moreover, while the invention has and hereinafter will be described in the context of fully functioning computers and computer systems, those skilled in the art will appreciate that the various embodiments of the invention are capable of being distributed as a program product in a variety of forms, and that the invention applies equally regardless of the particular type of computer readable, signal bearing media used to actually carry out the distribution. Examples of signal bearing, computer readable media include but are not limited to tangible, recordable type media such as volatile and non-volatile memory devices, floppy and other removable disks, hard disk drives, magnetic tape, optical disks (e.g., CD-ROM's, DVD's, etc.), among others, and transmission type media such as digital and analog communication links.

In addition, various program code described hereinafter may be identified based upon the application or engine within which it is implemented in a specific embodiment of the invention. However, it should be appreciated that any particular program nomenclature that follows is used merely for convenience, and thus the invention should not be limited to use solely in any specific application or engine identified and/or implied by such nomenclature.

Furthermore, given the typically endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), it should be appreciated that the invention is not limited to the specific organization and allocation of program functionality described herein.

The various software components and resources illustrated in FIG. 1 may be implemented in a number of manners, including using various computer software applications, routines, components, programs, objects, modules, data structures and programs. Those skilled in the art will further recognize that the exemplary environments illustrated in FIG. 1 are not intended to limit the present invention. Indeed, those skilled in the art will recognize that other alternative hardware and/or software environments may be used without departing from the scope of the invention.

FIG. 2 is a flowchart 50 having steps suitable for execution by the healthcare coordination system of FIG. 1. The steps of the flowchart 50 more specifically show processes that may be accomplished by the server computer 30 to identify, or determine, and display healthcare providers appropriate for a cash paying user. Turning more particularly to the flowchart 50, the server computer 30 may receive a user preference at block 52 of FIG. 2. The user preference may be geographic, price and/or service-type in nature. For instance, a user may input to their computer 41 a zip code preference corresponding to their locality. In another or the same example, the user may input a price range and/or the type of medical service they desire. Other preferences may include biographical data pertaining to a potential healthcare provider. For example, a user preference may include a query directed towards the gender of or the university attended by a healthcare professional.

Where desired, the system may allow the user to scale, or weight, their submitted user preferences. This feature may be used to closely account for the relative value of one user preference as compared to another. For instance, the system 10 may allow a user to designate that a geographic region is “very important”, while a preference for Saturday hours is “not so important”. In response, the system 10 may attach a mathematical factor to the zip code preference, effectively skewing determinations towards providers located in the specified region.

At block 54, the system 10 may access the database 37 to match the user preference(s). The database 37 typically stores healthcare provider information for a plurality of healthcare providers. The healthcare provider information routinely comprises at least one of geographic data, service-type data, biographic data and pricing data for cash patients and for each healthcare provider. To promote efficient retrieval of a list of potential healthcare providers, the healthcare provider information is linked logically to the appropriate healthcare provider within the database 37 in any manner known in the art.

The provider determination program 45 may determine at block 56 at least one provider associated with the user preference. That is, the program 45 may match the user preferences to corresponding markers of the healthcare provider information. The matched healthcare provider information may then be correlated to the appropriate healthcare providers. For instance, user preferences may indicate a desire for a podiatrist located within a given area code. The system 10 may process the data in the database 37 at block 56 to determine at least one provider practicing podiatry within or proximate the specified area code.

At block 58, the providers determined at block 56 may be displayed or otherwise communicated to the user. For instance, a list of one or more appropriate providers may be displayed on an Internet web page. The web page may include links to the respective homepages of the provider(s). A group of determined providers may be listed in order of their relevance to the submitted user preferences. For instance, the provider having the best percentage of healthcare provider information data matching the user preference (and accounting for any weighting criteria) may be listed first. Where desired, color indicators may accompany the list of providers to help guide the users in their selection. For instance, an indicator, e.g., a displayed color, grade, score or percentage, may communicate to the user that a particular provider matched the user preferences within 97%, while the next listed provider matched scored 73%.

In another embodiment, a list of providers may be electronically mailed or sent via postal mail to the user. Where desired, an automated telephonic message conveying contact information pertaining to the providers may be communicated to the user.

As discussed herein, a user may be encouraged to print out the web page of a determined healthcare provider. The user may additionally print out and sign a disclaimer for the doctor and as a record of their proposed fees.

FIG. 3 shows steps suitable for execution by the system 10 of FIG. 1 for receiving and posting healthcare provider information in a searchable format to facilitate provider determination. At block 62 of FIG. 3, the server computer 30 may receive healthcare provider information uploaded from a provider computer 43. Exemplary healthcare provider information may include one or more of geographic data, service type data and pricing data for uninsured patients. Pricing data may include a range of prices for a number of specific services and for cash paying patients. Other healthcare provider information may be of a biographic nature. To facilitate entry of the healthcare provider information, healthcare providers may select entries from drop-down menus, or providers may alternatively type the information into provided data fields.

Where desired, the system 10 may itemize or limit the number of services provided by a particular healthcare provider. For instance, the system may store and subsequently search only services submitted in connection with a provider.

To assist the provider in submitting the healthcare provider information, the system 10 may generate and send to the healthcare provider a price/services comparison report showing the average prices for a service offered by other participating providers operating under any number of conditions, e.g., in the same geographic region. The healthcare provider may browse the report to finalize or update their own healthcare provider information.

At block 64, the system 10 may store within the database 37 the submitted healthcare provider information in a searchable format. For instance, the system may store the healthcare provider information in logical association with the respective healthcare provider.

The submitted healthcare provider information may be posted at block 66. For instance, the healthcare provider information may be stored within the server database 37 in such a manner that the provider determination program 45 may readily search the information to determine healthcare providers.

While the present invention has been illustrated by a description of various embodiments and while these embodiments have been described in considerable detail, it is not the intention of the Applicant to restrict, or in any way limit, the scope of the appended claims to such detail. For instance, the system of another embodiment may initiate a payment from a provider as a fee for being listed within the system. Another embodiment may provide paperwork to initiate a contract between the patient and the healthcare provider, even accepting prepayment on behalf of the provider. Alternatively, the system may assess a fee to the patient for use of the website. In such an instance, the patient may be provided only with general information about the provider (and not the provider's actual contact information) until payment has been received. In still another embodiment, the system may accommodate bidding between providers for a submitted user preference.

As such, additional advantages and modifications will readily appear to those skilled in the art. The invention in its broader aspects is therefore not limited to the specific details, representative apparatus and method, and illustrative example shown and described. Accordingly, departures may be made from such details without departing from the spirit or scope of Applicant's general inventive concept.

What is claimed is:

1. A method for facilitating healthcare services, the method comprising:
receiving from a cash paying user a user preference relating to the healthcare service;
accessing a database storing healthcare provider information for each of a plurality of healthcare providers willing to perform the healthcare service on a cash basis, wherein at least a subset of the healthcare provider information is correlated to the user preference and is particular to a cash-based transaction;
identifying a healthcare provider from among the plurality of healthcare providers using the user preference; and
displaying the identified healthcare provider to the user.

2. The method of claim 1, wherein receiving the user preference further comprises receiving a geographic-related preference.

3. The method of claim 1, wherein receiving the user preference further comprises receiving at least one preference from a group consisting of: a geographic, a price, a biographic and a service-related preference.

4. The method of claim 1, wherein accessing the database further includes accessing healthcare provider information comprising at least one of geographic data, service-type data, biographic data and pricing data for cash paying patients and for each of the plurality of healthcare providers.

5. The method of claim 1, wherein identifying the healthcare provider further includes correlating the user preference to the healthcare provider information.

6. The method of claim 1, wherein displaying the identifying healthcare provider further comprises displaying the identified healthcare provider on a networked device.

7. The method of claim 1, further comprising generating a disclaimer for execution by the user.

8. The method of claim 1, further comprising itemizing a plurality of healthcare services performed by each healthcare provider.

9. The method of claim 1, wherein displaying the identified healthcare provider further comprises providing a link to a website of the healthcare provider.

10. The method of claim 1, wherein displaying the identified healthcare provider further comprises providing an indicator relating to how closely the user preference correlates to the healthcare provider information.

11. The method of claim 1, further comprising assessing a fee to at least one of the plurality of healthcare providers.

12. The method of claim 1, further comprising initiating a contract between the user and the healthcare provider.

13. The method of claim 1, further comprising accepting prepayment on behalf of a provider.

14. The method of claim 1, wherein displaying the identified healthcare provider further comprises generating a profile for the healthcare provider.

15. The method of claim 1, further comprising mathematically weighting at least one of the user preference and the healthcare provider information.

16. A method for facilitating healthcare services, the method comprising:
receiving healthcare provider information for each of a plurality of healthcare providers that each provide the healthcare service on a cash basis;
storing the healthcare provider information in a retrievable format within a memory; and
posting the healthcare provider information.

17. The method of claim 16, wherein receiving the healthcare provider information further includes receiving healthcare provider information relating to at least one of geographic, service-type, biographic and pricing data for cash paying patients.

18. The method of claim 16, further comprising enabling an update by a healthcare provider of the healthcare provider information.

19. An apparatus, comprising:
a processor;
a memory accessible to the processor, the memory including a database storing healthcare provider information; and
program code executable by the processor and configured to initiate receiving a user preference relating to a healthcare service to be performed on a cash basis, accessing the database, correlating the user preference to the healthcare provider information and identifying the healthcare provider, and displaying the identified healthcare provider to the user.

20. The apparatus of claim 19, wherein the cash basis includes payment selected from a group consisting of at least one of: credit card, debit card, health savings account, check, coin and note tender.
